(19) United States
(12) Patent Application Publication
(10) Pub. No.: US 2026/0015146 A1
(43) Pub. Date: Jan. 15, 2026

(54) Single Use Pouch Device

(71) Applicant: Patricia Hernandez, Baldwin Park, CA (US)

(72) Inventor: Patricia Hernandez, Baldwin Park, CA (US)

(21) Appl. No.: 18/769,892

(22) Filed: Jul. 11, 2024

(51) Int. Cl. B65D 75/58; A61J 1/10; A61M 35/00; B65D 75/30

(52) U.S. Cl. CPC B65D 75/5816; A61M 35/003; B65D 75/30; B65D 75/5811; A61J 1/10; B65D 2221/00

(57) ABSTRACT

A single use pouch device for carrying and subsequently dispensing a single use volume of a viscous skin treatment includes a pouch which contains a viscous skin treatment. The pouch has a total fluid volume sufficient to contain a pre-determined volume of the viscous skin treatment to cover a user's hands and arms. A first opening means is integrated into the pouch which enables a corner of the pouch to be removed thereby enabling the viscous skin treatment to be dispensed through the removed corner in a controlled manner. A second opening means is integrated into the pouch which enables the pouch to be divided thereby enabling the viscous skin treatment to be removed from the pouch. In this way the user can remove a maximum volume of the viscous skin treatment from the pouch.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to pouch devices and more particularly pertains to a new pouch device for carrying and subsequently dispensing a single use volume of a viscous skin treatment. The device includes a pouch which contains a measured volume of a viscous skin treatment. The pouch has a first opening means for removing a corner of the pouch thereby enabling the viscous skin treatment to be dispensed through the corner in a controlled manner. The pouch has a second opening means for dividing the pouch in half thereby enabling the full volume of the viscous skin treatment to be removed from the pouch in a single motion.

### (2) Description of Related Art Including Information Disclosed Under 37 CFR 1.97 and 1.98

The prior art relates to pouch devices including a variety of single use pouch devices that each at least includes a pouch containing a single use volume of a fluid and an opening means integrated into the pouch for dispensing the single use volume of the fluid. In certain instances the pouch is structured to define a neck which is exposed when the pouch is opened to enable the fluid to be dispensed through the neck in a controlled manner. In no instance does the prior art disclose a single use pouch device that includes a pouch which contains a viscous skin treatment and first opening means integrated into the pouch for removing a corner of the pouch to dispense the viscous skin treatment in a controlled manner and a second opening means integrated into the pouch for dividing the pouch in half to remove the full volume of the viscous skin treatment in a single motion.

### BRIEF SUMMARY OF THE INVENTION

An embodiment of the disclosure meets the needs presented above by generally comprising a pouch which contains a viscous skin treatment. The pouch has a total fluid volume sufficient to contain a pre-determined volume of the viscous skin treatment to cover a user's hands and arms. A first opening means is integrated into the pouch which enables a corner of the pouch to be removed thereby enabling the viscous skin treatment to be dispensed through the removed corner in a controlled manner. A second opening means is integrated into the pouch which enables the pouch to be divided thereby enabling the viscous skin treatment to be removed from the pouch. In this way the user can remove a maximum volume of the viscous skin treatment from the pouch.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a phantom perspective view of a single use pouch device according to an embodiment of the disclosure.

FIG. 2 is a front view of an embodiment of the disclosure showing a pouch being opened along a first opening means.

FIG. 3 is a front view of an embodiment of the disclosure showing a pouch being opened along a second opening means.

FIG. 4 is a perspective in-use view of an embodiment of the disclosure showing a viscous skin treatment being dispensed from a pouch.

### DETAILED DESCRIPTION OF THE INVENTION

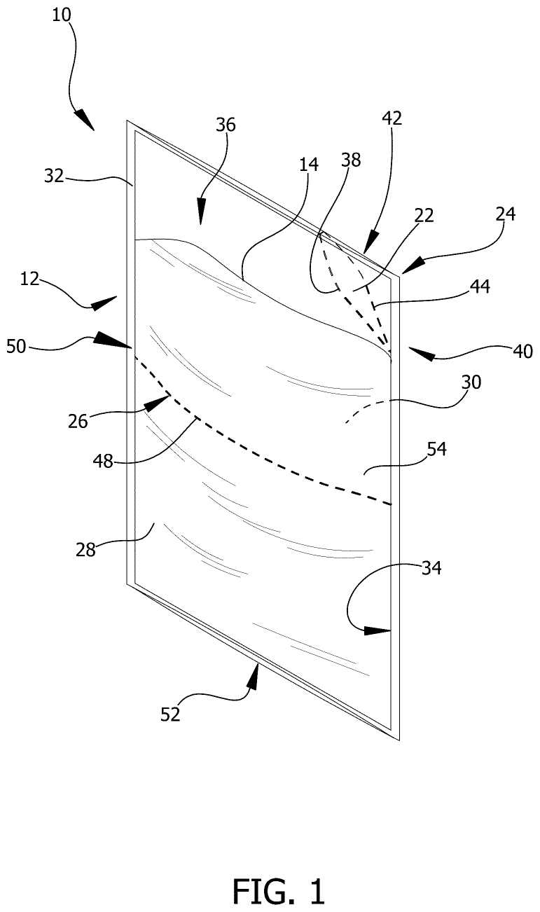

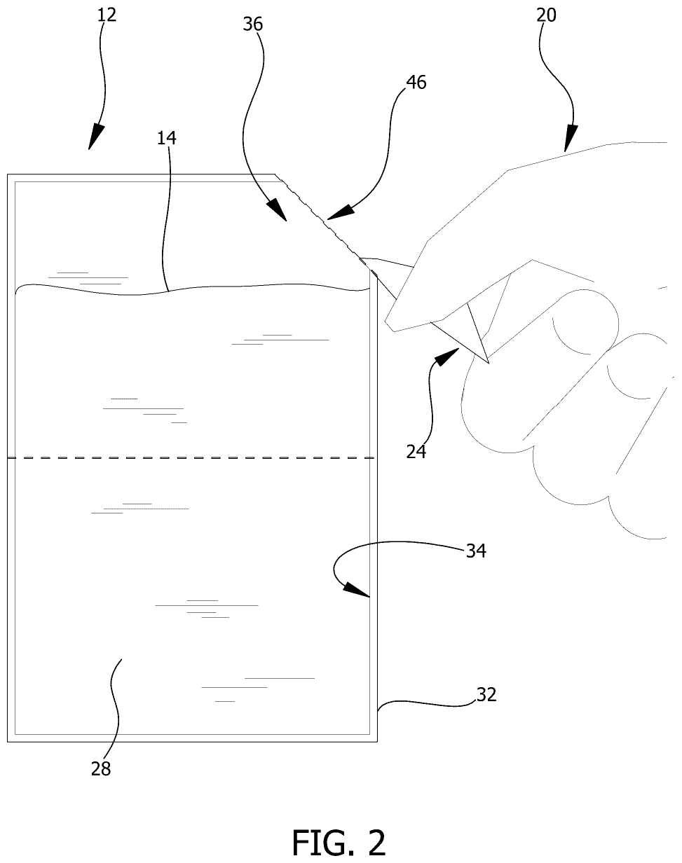

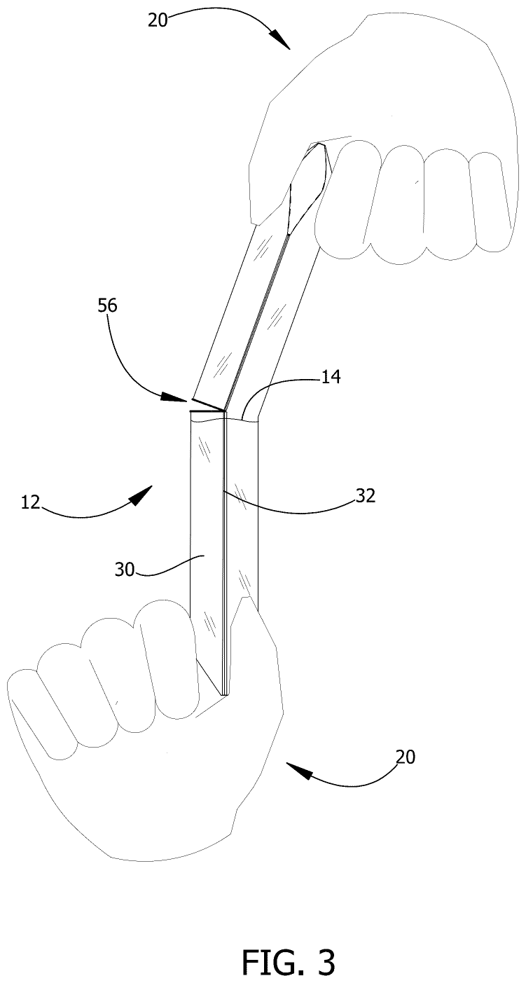

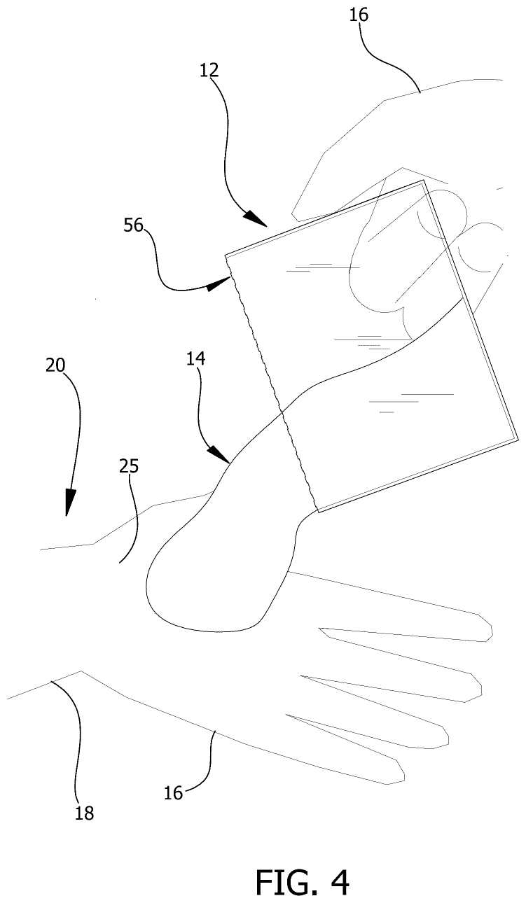

With reference now to the drawings, and in particular to FIGS. 1 through 4 thereof, a new pouch device embodying the principles and concepts of an embodiment of the disclosure and generally designated by the reference numeral 10 will be described.

As best illustrated in FIGS. 1 through 4, the single use pouch device 10 generally comprises a pouch 12 which is comprised of a fluid impermeable material, including but not being limited to foil or plastic film, thereby enabling the pouch 12 to contain a viscous skin treatment 14. The viscous skin treatment 14 may be skin lotion or other type of viscous skin treatment that is commonly applied to human skin for general purposes. The pouch 12 has a total fluid volume which is sufficient to contain a pre-determined volume of the viscous skin treatment 14. Furthermore, the pre-determined volume of the viscous skin treatment 14 is sufficient to cover the hands 16 and arms 18 of a user 20. In this way a single use volume of the viscous skin treatment 14 can be carried in the user's purse, for example, rather than requiring the user 20 to carry an entire container of the viscous skin treatment 14. Additionally, the pouch 12 may have a fluid volume ranging between approximately 15.0 ml and 20.0 ml.

The pouch 12 has a first opening means 22 integrated into the pouch 12 which enables a corner 24 of the pouch 12 to be removed thereby enabling the viscous skin treatment 14 to be dispensed through the removed corner 24. In this way the pouch 12 enables the viscous skin treatment 14 to be applied to the user's skin 25. Furthermore, the pouch 12 has a second opening means 26 integrated into the pouch 12 which enables the pouch 12 to be divided thereby enabling the viscous skin treatment 14 to be removed from the pouch 12. In this way the pouch 12 enables the user 20 to remove a maximum volume of the viscous skin treatment 14 from the pouch 12.

The pouch 12 has a front wall 28 and a back wall 30 and a perimeter edge 32 and each of the front wall 28 and the back wall 30 has a bonding point 34 which extends around the perimeter edge 32. Furthermore, the front wall 28 and the back wall 30 is bonded together at the bonding point 34 thereby enabling the front wall 28 and the back wall 30 to define an interior space 36 of the pouch 12 which contains the viscous skin treatment 14. The front wall 28 has a first perforation 38 which angles between a first lateral side 40 of the perimeter edge 32 and a top side 42 of the perimeter edge 32 and the back wall 30 has a second perforation 44 which is aligned with the first perforation 38. Additionally, the first perforation 38 and the second perforation 44 define the first opening means 22. Each of the front wall 28 and the back wall 30 are comprised of a semi-rigid material thereby enabling each of the front wall 28 and the back wall 30 to be breakable along the first perforation 38 and the second perforation 44. The front wall 28 is breakable along the first perforation 38 and the back wall 30 is breakable along the second perforation 44 thereby enabling an opening 46 to be defined between the first perforation 38 and the second perforation 44 for dispensing the viscous skin treatment 14. In this way the user 20 can squeeze the pouch 12 to dispense the viscous skin treatment 14 through the opening 46 in a controlled manner.

The front wall 28 has a third perforation 48 which extends between the first lateral side 40 of the perimeter edge 32 and a second lateral side 50 of the perimeter edge 32 and the third perforation 48 is centrally located between the top side 42 of the perimeter edge 32 and a bottom side 52 of the perimeter edge 32. Additionally, the back wall 30 has a fourth perforation 54 which is aligned with the third perforation 48 and the third perforation 48 and the fourth perforation 54 define the second opening means 26. The front wall 28 is breakable along the third perforation 48 and the back wall 30 is breakable along the fourth perforation 54 thereby enabling an opening 56 to be defined between the third perforation 48 and the fourth perforation 54 for dispensing the viscous skin treatment 14. In this way the user 20 can dispense the full volume of the viscous skin treatment 14 in a single motion.

In use, the user 20 places one or more of the pouches 12 in storage, such as a pocket of their clothing or in a purse for example, when the user 20 wishes to have access to the viscous skin treatment 14 during the course of the day. The pouch 12 is breakable along the first perforation 38 and the second perforation 44 to remove the respective corner 24 from the pouch 12. In this way the user 20 is enabled to dispense the viscous skin treatment 14 from the pouch 12 in a controlled manner by squeezing the pouch 12. The pouch 12 can be breakable along the third perforation 48 and the fourth perforation 54 rather than along the first perforation 38 and the second perforation 44 when the user 20 wishes to divide the pouch 12 in half. In this way the user 20 is enabled to dispense the full volume of the viscous skin treatment 14 in a single motion. Furthermore, the pouch 12 facilitates the user 20 to carry a single use volume of the viscous skin treatment 14 instead of carrying a full sized bottle of lotion, for example. In this way storage space is conserved for the user 20 thereby enabling the user 20 to carry a sufficient volume of the viscous skin treatment more efficiently 14 to meet their daily needs. The pouch 12 is discarded when the full volume of the viscous skin treatment 14 has been dispensed from the pouch 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A single use pouch device comprising:
a pouch being comprised of a fluid impermeable material thereby enabling said pouch to contain a viscous skin treatment, said pouch having a total fluid volume which is sufficient to contain a pre-determined volume of said viscous skin treatment wherein said pre-determined volume of said viscous skin treatment is configured to be sufficient to cover a user's hands and arms, said pouch having a first opening means being integrated into said pouch which enables a corner of said pouch to be removed thereby enabling said viscous skin treatment to be dispensed through said removed corner wherein said pouch is configured to enable said viscous skin treatment to be applied to the user's skin, said pouch having a second opening means being integrated into said pouch, said second opening being positioned at a middle of said pouch which enables said pouch to be divided in half thereby enabling said viscous skin treatment to be removed from said pouch wherein said pouch is configured to enable the user to remove a maximum volume of said viscous skin treatment from said pouch.

2. The device according to claim 1, wherein:
said pouch has a front wall and a back wall and a perimeter edge;
each of said front wall and said back wall has a bonding point which extends around said perimeter edge;
said front wall and said back wall being bonded together at said bonding point thereby enabling said front wall and said back wall to define an interior space of said pouch which contains said viscous skin treatment;
said front wall has a first perforation which angles between a first lateral side of said perimeter edge and a top side of said perimeter edge; and
said back wall has a second perforation which is aligned with said first perforation;
said first perforation and said second perforation defines said first opening means;
said front wall has a third perforation which extends between said first lateral side of said perimeter edge and a second lateral side of said perimeter edge;
said third perforation is centrally located between said top side of said perimeter edge and a bottom side of said perimeter edge;
said back wall has a fourth perforation which is aligned with said third perforation; and
said third perforation and said fourth perforation defines said second opening means.

3. The device according to claim 2, wherein said front wall is breakable along said first perforation and said back wall is breakable along said second perforation thereby enabling an opening to be defined between said first perforation and said second perforation for dispensing said viscous skin treatment.

4. The device according to claim 2, wherein said front wall is breakable along said third perforation and said back wall is breakable along said fourth perforation thereby enabling an opening to be defined between said third perforation and said fourth perforation for dispensing said viscous skin treatment.

5. A single use pouch device comprising:
a pouch being comprised of a fluid impermeable material thereby enabling said pouch to contain a viscous skin treatment, said pouch having a total fluid volume which is sufficient to contain a pre-determined volume of said viscous skin treatment wherein said pre-determined volume of said viscous skin treatment is configured to be sufficient to cover a user's hands and arms, said pouch having a first opening means being integrated into said pouch which enables a corner of said pouch to be removed thereby enabling said viscous skin treatment to be dispensed through said removed corner wherein said pouch is configured to enable said viscous skin treatment to be applied to the user's skin, said pouch having a second opening means being integrated into said pouch, said second opening being positioned at a middle of said pouch which enables said pouch to be divided in half thereby enabling said viscous skin treatment to be removed from said pouch wherein said pouch is configured to enable the user to remove a maximum volume of said viscous skin treatment from said pouch, said pouch having a front wall and a back wall and a perimeter edge, each of said front wall and said back wall having a bonding point which extends around said perimeter edge, said front wall and said back wall being bonded together at said bonding point thereby enabling said front wall and said back wall to define an interior space of said pouch which contains said viscous skin treatment, said front wall having a first perforation which angles between a first lateral side of said perimeter edge and a top side of said perimeter edge, said back wall having a second perforation which is aligned with said first perforation, said first perforation and said second perforation defining said first opening means, each of said front wall and said back wall being comprised of a semi-rigid material thereby enabling said front wall to be breakable along said first perforation and enabling said back wall to be breakable along said second perforation thereby enabling an opening to be defined between said first perforation and said second perforation for dispensing said viscous skin treatment, said front wall having a third perforation which extends between said first lateral side of said perimeter edge and a second lateral side of said perimeter edge, said third perforation being centrally located between said top side of said perimeter edge and a bottom side of said perimeter edge, said back wall having a fourth perforation which is aligned with said third perforation, said third perforation and said fourth perforation defining said second opening means, said front wall being breakable along said third perforation and said back wall being breakable along said fourth perforation thereby enabling an opening to be defined between said third perforation and said fourth perforation for dispensing said viscous skin treatment.

6. A method of storing and subsequently dispensing a single-use volume of a viscous skin treatment, the steps of the method comprising:
carrying a pouch containing a measured volume of the viscous skin treatment which is determined to be sufficient to cover a user's hands and arms wherein said pouch is configured to enable a user to carry a single use volume of said viscous skin treatment rather than carrying an entire container of said viscous skin treatment;
breaking said pouch along a first opening means thereby enabling a corner of said pouch to be removed from said pouch thereby enabling said viscous skin treatment to be dispensed through said removed corner wherein said pouch is configured to enable a user to dispense said viscous skin treatment in a controlled manner; or
breaking said pouch along a second opening means rather than breaking said pouch along said first opening means, said second opening being positioned at a middle of said pouch which enables said pouch to be divided in half thereby enabling said pouch to be divided in half thereby enabling a maximum volume of said viscous skin treatment to be removed from said pouch wherein said pouch is configured to enable the user to remove the full volume of said viscous skin treatment in a single motion.
